(19) United States
(12) Patent Application Publication
(10) Pub. No.: US 2019/0244711 A1
(43) Pub. Date: Aug. 8, 2019

(54) SYSTEM AND METHOD FOR TIMING DELIVERY OF QUERIES TO TELEHEALTH PATIENTS

(71) Applicant: KONINKLIJKE PHILIPS N.V., EINDHOVEN (NL)

(72) Inventors: Rony CALO, Amsterdam (NL); Aart Tijmen VAN HALTEREN, Geldrop (NL)

(21) Appl. No.: 16/312,092

(22) PCT Filed: Jun. 19, 2017
(86) PCT No.: PCT/EP2017/064878

§ 371 Date: Dec. 20, 2018

Related U.S. Application Data
(60) Provisional application No. 62/356,572, filed on Jun. 30, 2016.

(51) Int. Cl. G16H 50/30; G06F 16/9535; G06F 16/9538; G06F 16/25; G16H 10/60; G16H 50/20

(52) U.S. Cl. CPC G16H 50/30; G06F 16/9535; G16H 50/20; G06F 16/252; G16H 10/60; G06F 16/9538

(57) ABSTRACT

The present system is configured to use vital signs levels, psychological information, and engagement information that is typically collected within telehealth programs, along with responses to queries presented to a subject via query interfaces, to: compute baseline well-being vital signs ranges for the subject; monitor the subject's physiological and emotional well-being relative to the baseline well-being vital signs ranges; withhold queries (presented via the query interfaces) selected by telehealth professionals for the subject in a database until the vital signs of the subject are within baseline ranges; and present the queries (via a query interface) to the subject responsive to the vital signs of the subject being within the baseline well-being ranges.

### FIELD OF THE INVENTION

The present disclosure pertains to a telemonitoring system configured to interact with a subject.

### BACKGROUND OF THE INVENTION

Typically, patients in home telehealth programs are required to input data about themselves to their care providers on a periodic basis. Requests for such information are usually sent to patients at predetermined times, in order to assess medical conditions experienced by the patients over time.

### SUMMARY OF THE INVENTION

Accordingly, one or more aspects of the present disclosure relate to a telemonitoring system configured to interact with a subject. The system comprises an input unit configured to receive one or more signals generated from one or more sensors, additionally or alternatively, the system comprises one or more sensors. The system further comprises a user interface, one or more hardware processors, and/or other components. The one or more sensors are configured to generate output signals conveying information related to one or more vital signs of the subject. The user interface is configured to present the user with one or more query interfaces and facilitate entry and/or selection of responses to queries presented by the query interfaces. The one or more hardware processors are configured by machine readable instructions to: cause presentation of one or more learning phase query interfaces to the subject via the user interface, the one or more learning phase query interfaces comprising queries related to a well-being of the subject; determine, based on the output signals, first levels of the one or more vital signs for periods of time that correspond to a timing of responses to the queries of the one or more learning phase query interfaces by the subject; determine well-being ranges for the one or more vital signs of the subject based on (1) the first levels of the one or more vital signs, and (2) information conveyed by the responses to the queries of the one or more learning phase query interfaces; receive a request, for instance from a telehealth professional or a database (e.g. an electronic database, a memory), for presentation of a subject evaluation query interface to the subject, the subject evaluation query interface comprising queries related to a medical condition experienced by the subject; determine, based on the output signals, second levels of the one or more vital signs of the subject; and responsive to the second levels of the one or more vital signs being within the well-being ranges, cause presentation of the subject evaluation query interface to the subject via the user interface.

Another aspect of the present disclosure relates to a method for facilitating interaction between a subject and an interaction system. The system comprises an input unit for receiving sensor signal(s) from one or more sensors, and/or comprises one or more sensors, a user interface, one or more hardware processors, and/or other components. The method comprises: receiving from or generating with the one or more sensors, output signals conveying information related to one or more vital signs of the subject; causing presentation, with the one or more hardware processors, of one or more learning phase query interfaces to the subject via the user interface, the one or more learning phase query interfaces comprising queries related to a well-being of the subject; determining, with the one or more hardware processors based on the output signals, first levels of the one or more vital signs for periods of time that correspond to a timing of responses to the queries of the one or more learning phase query interfaces by the subject; determining, with the one or more hardware processors, well-being ranges for the one or more vital signs of the subject based on (1) the first levels of the one or more vital signs, and (2) information conveyed by the responses to the queries of the one or more learning phase query interfaces; receiving, with the one or more hardware processors, a request, for instance from a telehealth professional or a database (e.g. an electronic database a memory), for presentation of a subject evaluation query interface to the subject, the subject evaluation query interface comprising queries related to a medical condition experienced by the subject; determining, with the one or more hardware processors based on the output signals, second levels of the one or more vital signs of the subject; and responsive to the second levels of the one or more vital signs being within the well-being ranges, causing, with the one or more hardware processors, presentation of the subject evaluation query interface to the subject via the user interface.

Still another aspect of present disclosure relates to a telemonitoring system configured to interact with a subject. The system comprises: means for receiving and/or generating output signals conveying information related to one or more vital signs of the subject; means for presenting the user with one or more query interfaces and facilitating entry and/or selection of responses to queries presented by the query interfaces; means for causing presentation of one or more learning phase query interfaces to the subject via the means for presenting, the one or more learning phase query interfaces comprising queries related to a well-being of the subject; means for determining, based on the output signals, first levels of the one or more vital signs for periods of time that correspond to a timing of responses to the queries of the one or more learning phase query interfaces by the subject; means for determining well-being ranges for the one or more vital signs of the subject based on (1) the first levels of the one or more vital signs, and (2) information conveyed by the responses to the queries of the one or more learning phase query interfaces; means for receiving a request, for instance from a telehealth professional or a database (e.g. an electronic database, a memory), for presentation of a subject evaluation query interface to the subject, the subject evaluation query interface comprising queries related to a medical condition experienced by the subject; means for determining, based on the output signals, second levels of the one or more vital signs of the subject; and means for, responsive to the second levels of the one or more vital signs being within the well-being ranges, causing presentation of the subject evaluation query interface to the subject via the means for presenting.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1A illustrates a telemonitoring system configured to interact with a subject.

FIG. 1B illustrates sensors included in the telemonitoring system.

FIG. 1C illustrates a computing device included in the telemonitoring system formed as a laptop, a desktop computer, a smartphone, and/or a tablet computer.

FIG. 2 illustrates a method for facilitating interaction between a subject and a telemonitoring system.

### DETAILED DESCRIPTION OF EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Subjects in home telehealth programs are often required to provide data and information about themselves to their care providers. Such data and information can be provided, for example, via questionnaires and/or queries through computing devices associated with the subject. The questionnaires and/or queries typically ask about health behaviors performed by the patients (e.g., exercise), adherence to prescribed medication regimens, overall confidence levels, quality of life, and/or other factors. However, these questionnaires and/or queries are sent to subjects at predetermined times, without regard for a physiological and/or emotional well-being of an individual subject. This often results in unreliable responses to queries asked of a subject. The unreliable responses may be purposeful or accidental, but in either case tend to increase with fluctuations in a subject's well-being over time. Response reliability increases when subjects answer questions at times when they are physically and/or emotionally stable. Responding to questions at physically and/or emotionally stable times, and/or when the physical and/or emotional status of the subject is the same as when questions were answered previously, increases the reliability of answers and increases the detectability of changes in the responses.

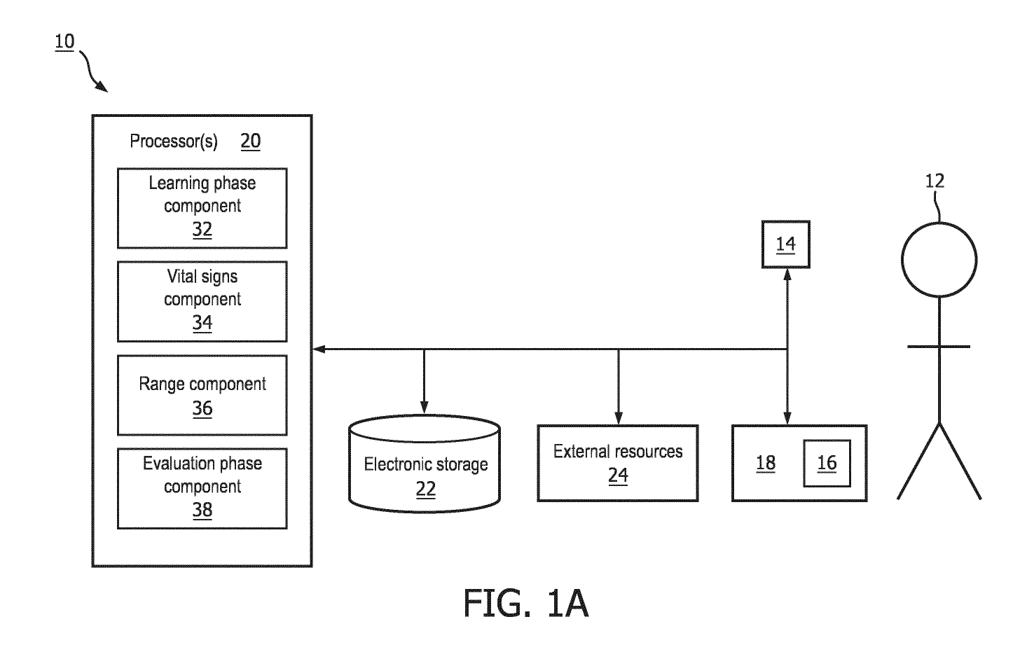

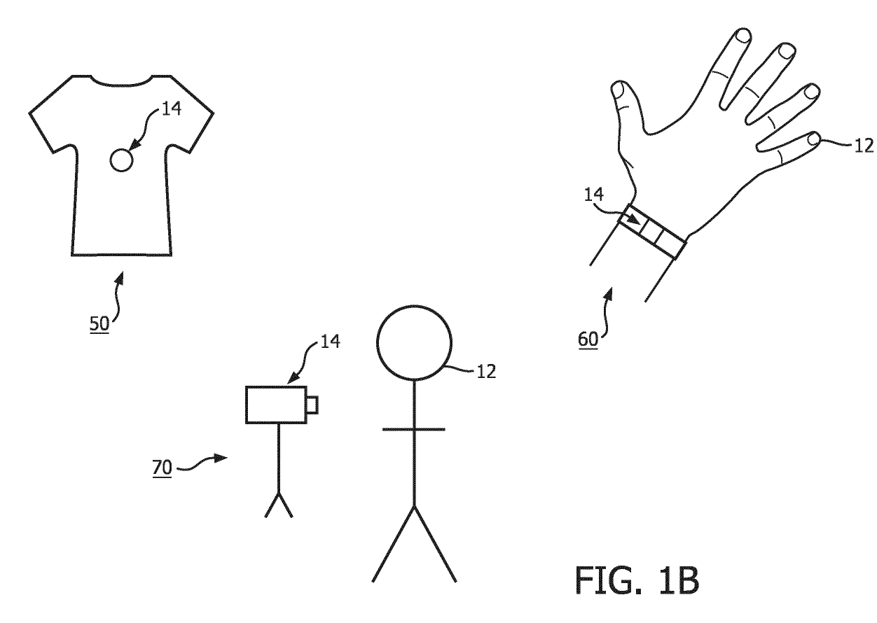

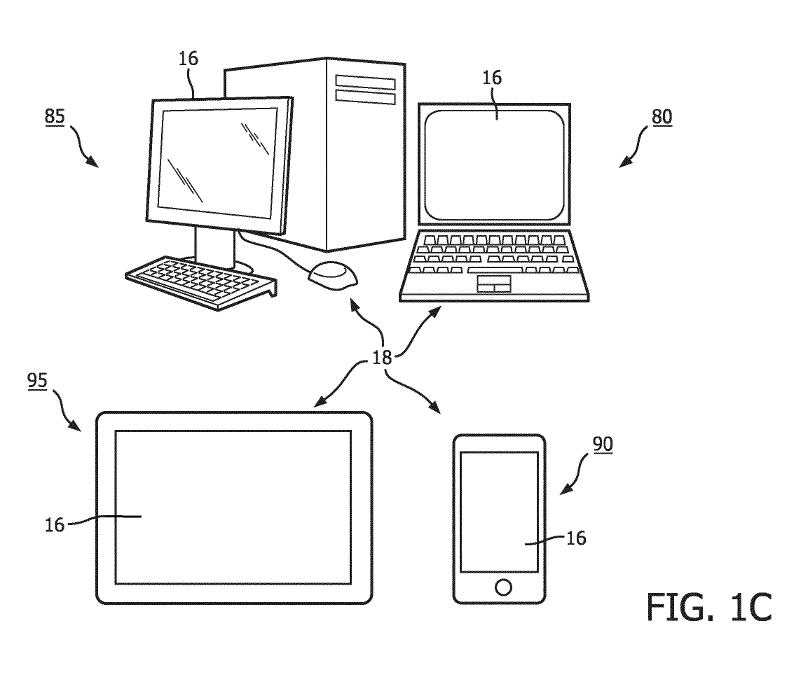

FIG. 1A illustrates a telemonitoring system 10 configured to interact with a subject 12. System 10 increases the reliability of information collected from subjects (e.g., subject 12) in telehealth programs by querying subject 12 at times when subject 12 is physically and/or emotionally stable according to the system's observations. System 10 is configured to use vital signs levels, psychological information and engagement information that is typically collected within telehealth programs, along with responses to queries presented to subject 12 via query interfaces (described below) and/or other information to: (1) determine baseline well-being vital signs ranges for subject 12; (2) monitor the physiological and emotional well-being of subject 12 relative to the baseline well-being vital signs ranges; (3) withhold queries (presented via the query interfaces) selected by telehealth professionals (e.g., doctors, nurses, care coordinators, and/or other caregivers) for subject 12 (e.g., in an electronic database) until the vital signs of subject 12 are within the baseline ranges; (4) present the queries (via a query interface) to subject 12 responsive to the vital signs of subject 12 being within the baseline well-being ranges; and/or perform other operations.

System 10 operates in phases including a learning phase, an evaluation phase, and/or other phases. In the learning phase, system 10 learns how the vital signs of subject 12 change responsive to physical, psychological, and/or other changes in subject 12. System 10 determines (e.g., baseline) well-being ranges for the vital signs that reflect a subject's normal and/or typical psychological and/or physical condition. In the evaluation phase, system 10 presents queries from telehealth professional to subject 12 when levels of the vital signs of subject 12 are within the well-being ranges, and withholds the queries if one or more levels of the vital signs are not within the well-being ranges. In some embodiments, system 10 comprises sensors 14, a computing device 18, one or more processors 20, electronic storage 22, external resources 24, and/or other components.

Sensors 14 are configured to generate output signals conveying information related to one or more vital signs of subject 12. In some embodiments, sensors 14 include but are not limited to equipment used in hospitals, doctor's offices, and/or other medical facilities, in the home of subject 12, and/or in other locations to monitor vital signs and/or other physiological information (e.g., pulse rate monitors, blood pressure monitors, blood oxygenation monitors, glucose monitors, weight scales, thermometers, electrocardiogram (EKG) equipment, childbirth labor contraction monitors, etc.), test equipment (e.g., imaging equipment such as an MRI and/or an x-ray machine, an ultrasound, electroencephalogram (EEG) equipment, etc.), equipment for treating subject 12 (e.g., respirators/ventilators, light therapy devices, etc.), devices for entering and/or selecting information (e.g., desktop computers, laptop computers, tablet computers, smartphones, cameras, video equipment, etc.) associated with subject 12, and/or other devices. Sensors 14 may comprise one or more sensors that measure vital signs directly. For example, sensors 14 may include electrodes configured to detect electrical activity in the heart of subject 12, light based sensors coupled with a finger of subject 12 configured to detect a pulse rate and/or a blood chemistry (e.g., an oxygen level) of subject 12, a blood pressure cuff configured to facilitate determination of the blood pressure of subject 12, a thermometer coupled to subject 12, and/or other sensors. Sensors 14 may comprise one or more sensors that generate output signals conveying information related to vital signs of subject 12 indirectly. For example, one or more sensors 14 may include a camera configured to generate an output based on images of subject 12 (e.g., a heart rate of subject 12, respiration of subject 12, a temperature of subject 12, an oxygen saturation level of subject 12, and/or other characteristics of subject 12). Although sensors 14 are illustrated in FIG. 1A at a single location near subject 12, this is not intended to be limiting. As shown in FIG. 1B, sensors 14 may include sensors disposed in a plurality of locations, such as for example, coupled 50 (in a removable manner) with clothing of subject 12, worn 60 by subject 12 (e.g., as a headband, wristband, a blood pressure cuff, a finger clip, etc.), positioned to point 70 at subject 12 (e.g., a camera), temporarily held and/or supported by subject 12 (e.g., a thermometer), and/or in other locations.

Returning to FIG. 1A, computing device 18 is configured to provide an interface between subject 12 and system 10. In some embodiments, computing device 18 is associated with subject 12 and/or other users. Computing device 18 is configured to provide information to and/or receive information from subject 12 and/or other users. Computing device 18 includes a user interface 16 and/or other components. User interface 16 is configured to present subject 12 with one or more query interfaces and facilitate entry and/or selection of responses to queries presented by the query interfaces (e.g., learning phase and/or evaluation query interfaces as described herein). A query interface may be an electronic view of a graphical user interface presented by user interface 16. The query interfaces may comprise queries (e.g., individual questions, a questionnaire and/or other queries) and/or other query information configured to provide information to subject 12 and/or cause subject 12 to provide information to system 10. In some embodiments, user interface 16 includes a plurality of separate interfaces associated with computing device 18, processor 20 and/or other components of system 10, for example.

In some embodiments, computing device 18 is configured to provide user interface 16, processing capabilities, databases, and/or electronic storage to system 10. As such, computing device 18 may include processors 20, electronic storage 22, external resources 24, sensors 14, and/or other components of system 10. In some embodiments, computing device 18 is connected to a network (e.g., the internet). In some embodiments, computing device 18 does not include processor 20, electronic storage 22, external resources 24, sensors 14, and/or other components of system 10, but instead communicates with these components via the network. The connection to the network may be wireless or wired. For example, processor 20 may be located in a remote server and may wirelessly cause display of the query interfaces via user interface 16 to subject 12 on computing device 18. As shown in FIG. 1C, in some embodiments, computing device 18 is a laptop 80, a desktop computer 85, a smartphone 90, a tablet computer 95, and/or other computing devices.

Examples of interface devices suitable for inclusion in user interface 16 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing device 18 includes a removable storage interface. In this example, information may be loaded into computing device 18 from removable storage (e.g., a smart card, a flash drive, a removable disk) that enables subject 12 and/or other users to customize the implementation of computing device 18. Other exemplary input devices and techniques adapted for use with computing device 18 and/or user interface 16 include, but are not limited to, an RS-232 port, RF link, an IR link, a modem (telephone, cable, etc.) and/or other devices.

Returning to FIG. 1A, processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1A as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, computing device 18, sensors 14, devices that are part of external resources 24, electronic storage 22, and/or other devices.)

In some embodiments, processor 20, sensors 14, external resources 24, computing device 18, electronic storage 22, and/or other components may be operatively linked via one or more electronic communication links For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with sensors 14, external resources 24, computing device 18, electronic storage 22, and/or other components according to a client/server architecture, a peer-to-peer architecture, and/or other architectures.

As shown in FIG. 1, processor 20 is configured via machine-readable instructions to execute one or more computer program components. The one or more computer program components may comprise one or more of a learning phase component 32, a vital signs component 34, a range component 36, an evaluation phase component 38, and/or other components. Processor 20 may be configured to execute components 32, 34, 36, and/or 38 by software; hardware; firmware; some combination of software, hardware, and/or firmware; and/or other mechanisms for configuring processing capabilities on processor 20.

It should be appreciated that although components 32, 34, 36, and 38 are illustrated in FIG. 1 as being co-located within a single processing unit, in embodiments in which processor 20 comprises multiple processing units, one or more of components 32, 34, 36, and/or 38 may be located remotely from the other components. The description of the functionality provided by the different components 32, 34, 36, and/or 38 described below is for illustrative purposes, and is not intended to be limiting, as any of components 32, 34, 36, and/or 38 may provide more or less functionality than is described. For example, one or more of components 32, 34, 36, and/or 38 may be eliminated, and some or all of its functionality may be provided by other components 32, 34, 36, and/or 38. As another example, processor 20 may be configured to execute one or more additional components that may perform some or all of the functionality attributed below to one of components 32, 34, 36, and/or 38.

Learning phase component 32 is configured to cause presentation of one or more learning phase query interfaces to subject 12 via user interface 16. The one or more learning phase query interfaces comprise queries (e.g., questions) related to a well-being of subject 12 and/or other queries. The well-being of subject 12 may be indicative of an overall physical condition of subject 12, the relative health and/or sickness of subject 12, psychological characteristics and/or status of subject 12, a level of engagement between subject 12 and system 10, a level of engagement between subject 12 and caregivers (e.g., telehealth professionals, family members, friends, etc.), social characteristics of subject 12, and/or other characteristics of subject 12. For example, learning phase component 32 may configured to cause presentation of one or more learning phase query interfaces that ask questions about how much subject 12 exercises, how much subject 12 sleeps, symptoms of one or more illnesses subject 12 may be experiencing, symptoms of and/or or changes in symptoms of one or more chronic diseases experienced by subject 12, whether subject 12 is feeling good, anxious, or depressed, whether subject 12 has been taking medication as instructed, and/or any other questions (this example list is not intended to be limiting) relevant to the well-being of subject 12. In some embodiments, learning phase component 32 is configured to determine information related to the well-being of subject 12 by obtaining information from one or more electronic medical records (EMR) associated with subject 12. For example, EMR information related a deterioration and/or improvement in medical conditions experienced by subject 12 may be obtained. The EMR associated with subject 12 may be stored in a database that is part of external resources 24 and/or be stored in other locations.

In some embodiments, learning phase component 32 is configured to obtain psychological information associated with subject 12 as part of the learning phase. The psychological information may be obtained from electronic medical records associated with subject 12 (e.g., from a database included in external resources 24), received from telehealth professionals (e.g., via a computing device 18 associated with the telehealth professionals), received from subject 12 via a query interface presented via user interface 16, and/or obtained by other methods. The psychological information may include information related to a mental capacity of subject 12, moods of subject 12, an overall demeanor of subject 12, mental illness experienced by subject 12, abnormal and/or other behavior of subject 12, psychiatric problems (e.g., depression, anxiety, etc.) experienced by subject 12, and/or other psychological information. For example, the psychological information may indicate when subject 12 is feeling normal, feeling anxious, feeling depressed and/or other having other feelings.

In some embodiments, learning phase component 32 is configured to determine engagement information (e.g., first engagement information) indicative of a level of engagement of subject 12 with telemonitoring system 10, engagement information (e.g., second engagement information) indicative of a level of engagement of subject 12 with a care team associated with subject 12, and/or other engagement information as part of the learning phase. In some embodiments, learning phase component 32 is configured such that determining engagement information (e.g., the first engagement information) includes asking subject 12 engagement related queries via a query interface and analyzing information in responses to the queries, detecting interaction patterns of the user with computing device 18 and/or user interface 16, and/or determining other information. In some embodiments, detecting interaction patterns includes determining whether subject 12 uses computing device 18 on a given day, determining how long subject 12 uses computing device 18 on a given day and/or during an individual interaction session, determining whether subject 12 responds to queries presented by a learning phase query interface immediately after the learning phase query interface is presented to subject 12 or whether subject 12 waits to respond to the queries presented, determining whether subject 12 complies with directions to measure vital signs, and/or other interaction pattern detection.

In some embodiments, learning phase component 32 is configured such that determining engagement information (e.g., second engagement information) indicative of a level of engagement of subject 12 with a care team associated with subject 12 includes asking subject 12 engagement related queries via a query interface and analyzing information in responses to the queries, determining whether subject 12 communicates with the care team via computing device 18, determining a regularity and/or irregularity of communications from subject 12 (e.g., to a telehealth call/communication center via information in a call center database included in external resources 24), determining whether subject 12 attends scheduled medical appointments (e.g., based on information in an EMR associated with subject 12 stored in a database that is part of external resources 24), and/or other engagement determinations.

Vital signs component 34 is configured to determine vital signs of subject 12. Vital signs component 34 is configured to determine vital signs of subject 12 based on the output signals from sensors 14 and/or other information. In some embodiments, vital signs component 34 is configured to determine multiple different levels of the one or more vital signs at different times. In some embodiments, determined vital signs levels may include learning phase vital signs levels, evaluation phase vital signs levels, current vital signs levels, and/or other vital signs levels, for example. Vital signs component 34 may determine first, second, third, fourth (and so on) levels of the vital signs. The various levels (e.g., first, second, third, fourth, etc.) of the vital signs may be determined at predetermined times (e.g., minute by minute, hourly, daily, weekly, monthly, etc.); responsive to a request from subject 12, a telehealth professional and/or other users; responsive to and/or during presentation of a learning phase query interface to subject 12; responsive to and/or during the obtaining of the psychological information associated with subject 12; responsive to and/or during the determination of the engagement information; responsive to receiving a receive a request from a telehealth professional for presentation of a subject evaluation query interface to subject 12, and/or at other times.

Vital signs component 34 is configured such that first current levels (for example) of the one or more vital signs are determined for periods of time that correspond to a timing of responses to the queries of the one or more learning phase query interfaces by subject 12. Vital signs component 34 is configured such that the levels of the vital signs determined for the periods of time that correspond to a timing of responses to the queries correspond to information conveyed in the responses by subject 12. Current levels of the one or more vital signs may be determined for periods of time that correspond to a timing of obtaining the psychological information. For example, vital signs component 34 is configured to determine vital signs that correspond to periods of time when subject 12 is feeling and/or felt good, vital signs that correspond to periods of time when subject 12 is feeling and/or felt anxious, vital signs that correspond to periods of time when subject 12 feels and/or felt depressed, and/or vital signs that correspond to periods of time when subject 12 feels and/or felt other feelings. Current levels of the one or more vital signs may be determined for periods of time that correspond to a timing of determining the (e.g., first and second) engagement information described above. For example, vital signs component 34 is configured to determine vital signs that correspond to periods of time when subject 12 was engaged with system 10 and/or the caregivers of subject 12, vital signs that correspond to periods of time when subject 12 was not engaged with system 10 and/or the caregivers, and/or other vital signs. In some embodiments, the vital signs that are determined for periods of time that correspond to a timing of responses to the queries of the one or more learning phase query interfaces, the vital signs that correspond to a timing of obtaining the psychological information, the vital signs that correspond to a timing of determining the (e.g., first and second) engagement information, and/or other vital signs may be and/or be included in the learning phase vital signs.

Range component 36 is configured to determine well-being (e.g., baseline) ranges for the one or more vital signs of subject 12. The (e.g., baseline) well-being ranges for the vital signs reflect a normal and/or typical psychological and/or physical condition of subject 12. Range component 36 is configured to determine the well-being ranges based on the levels of the one or more vital signs, information conveyed by the responses to the queries of the one or more learning phase query interfaces, the obtained psychological information, the engagement information (e.g., first engagement information) indicative of a level of engagement of subject 12 with telemonitoring system 10, the engagement information (e.g., second engagement information) indicative of a level of engagement of subject 12 with a care team associated with subject 12, and/or other information.

In some embodiments, range component 36 is configured such that determining the well-being ranges includes determining how the vital signs of subject 12 change in response to psychological and/or physical deterioration and/or improvement in subject 12. In some embodiments, range component 36 is configured to determine the physical and/or psychological condition of subject 12 over a specified period of time (e.g., based on the information determined and/or obtained by learning phase component 32 as described above) and correlate changes in the determined vital signs (e.g., by vital signs component 34) over this specified period of time to changes in the physical and/or psychological condition of subject 12. Range component 36 is configured to determine how (e.g., percentage) changes in vital signs (e.g., an increase in blood pressure) correspond to changes (e.g., a deterioration) in the psychosocial well-being of subject 12, determine how (e.g., percentage) changes in vital signs correspond to changes (e.g., a deterioration) in the physical well-being of subject 12, and determine the (e.g., baseline) well-being ranges based on these correlations and/or other information. By way of a non-limiting example, such correlations may be determined by computing changes in individual vital signs between two points of time (e.g., subtracting the value of a given vital sign at one point in time from the value of the given vital sign an another point in time), computing changes in individual wellbeing factors between the same two points of time (e.g., subtracting the value of a given wellbeing factor at one point in time from the value of the given wellbeing factor at the other point in time), performing regression analyses using the corresponding changes in vital signs and wellbeing factors, and/or performing other operations. The correlations described above facilitate determination of the psychosocial and/or physical well-being of subject 12 based on vital signs alone, for example.

In some embodiments, range component 36 is configured to re-determine the well-being ranges for the one or more vital signs (a) at predetermined minute by minute, daily, weekly, monthly, or yearly intervals; (b) responsive to a request by the telehealth professional; (c) responsive to a predetermined change in the medical condition experienced by subject 12, and/or at other times. By way of a non-limiting example, range component 36 is configured to re-determine the well-being ranges because patients with chronic diseases often experience deterioration in their conditions over time. This deterioration is reflected in changes in their “normal” vital signs ranges and as such also influences their (e.g., baseline) well-being vital signs ranges.

Evaluation phase component 38 is configured to receive a request from a telehealth professional for presentation of a subject evaluation query interface to subject 12. The subject evaluation query interface comprises queries related to a medical condition experienced by subject 12 and/or other information. For example, the queries may ask about symptoms of the chronic disease experienced by subject 12, whether medication, exercises, and/or other treatments have been followed by the patient and whether they have been effective and/or ineffective, how the treatments have been experienced by the patient and reasons they have or have not been followed by the patient, and/or other characteristics of subject 12.

Evaluation phase component 38 is configured to obtain (from vital signs component 34) and/or determine (based on the output signals from sensors 14 and/or other information), evaluation phase vital signs levels of the vital signs of subject 12. The evaluation phase vital signs levels may be and/or include current (e.g., second relative to the example above) levels of the one or more vital signs of subject 12 and/or other vital signs levels. The current levels of the vital signs may be obtained and/or determined by evaluation phase component 38 responsive to receiving the request from the telehealth professional for presentation of the subject evaluation query interface, at a predetermined minute by minute, hourly, daily, weekly, monthly, etc. time (e.g., programed at manufacture of system 10), responsive to subject 12 and/or a caregiver conducting vital signs measurements, and/or at other times. Responsive to the current levels of the one or more vital signs being within the well-being ranges (indicating subject 12 is feeling and/or behaving “normally”, and/or the vital signs of subject 12 are within “normal” and/or “typical” ranges), evaluation phase component 38 is configured to cause presentation of the subject evaluation query interface to subject 12 via user interface 16. Responsive to one or more of the current levels of the one or more vital signs being outside the well-being ranges (indicating subject 12 is not feeling and/or behaving “normally”, and/or the vital signs of subject 12 are not within “normal” and/or “typical” ranges which increases the likelihood of unreliable responses to the queries), evaluation phase component 38 is configured to withhold the subject evaluation query interface from subject 12 until the current levels of the one or more vital signs are within the well-being ranges.

In some embodiments, the requests for presentation of individual subject evaluation query interfaces and/or the subject evaluation query interfaces and the queries themselves are electronically stored in electronic storage 22, in a database that is part of external resources 24, and/or in other locations. Responsive to the current levels of the one or more vital signs being within the well-being ranges (indicating subject 12 is feeling and/or behaving “normally”, and/or the vital signs of subject 12 are within “normal” and/or “typical” ranges), evaluation phase component 38 is configured to obtain a given requested subject evaluation query interface from electronic storage and cause presentation of the subject evaluation query interface to subject 12 via user interface 16. Responsive to one or more of the current levels of the one or more vital signs being outside the well-being ranges (indicating subject 12 is not feeling and/or behaving “normally”, and/or the vital signs of subject 12 are not within “normal” and/or “typical” ranges), evaluation phase component 38 is configured to maintain the subject evaluation query interface and the associated queries in electronic storage until the current levels of the one or more vital signs are within the well-being ranges (this also applies to requests for presentation of subject evaluation user interfaces received while system 10 is operating in the learning phase described above).

As described above, system 10 (e.g., learning phase component 32, vital signs component 34, range component 36) is configured to determine the psychosocial and/or physical well-being of subject 12 based on the levels of the vital signs. System 10 (e.g., learning phase component 32, vital signs component 34, range component 36) determines when the well-being of subject 12 has changed (e.g., deteriorated). Evaluation phase component 38 determines when to cause presentation of subject evaluation query interfaces based on the well-being of subject 12 (e.g., when the levels of the vital signs indicate subject 12 is not well, evaluation phase component 38 does not cause presentation of the subject evaluation query interfaces). Evaluation phase component 38 is configured such that withheld subject evaluation query interfaces and their associated queries are not presented to subject 12 all at once responsive to the levels of the vital signs of subject 12 retuning to the (e.g., baseline) well-being ranges. Instead, evaluation phase component 38 determines an importance of individually requested subject evaluation query interfaces and/or the queries included in a requested subject evaluation query interface, and causes presentation of the formerly withheld subject evaluation query interfaces based on the determined importance. In some embodiments, evaluation phase component 38 is configured to determine the importance based on information provided with the request by the telehealth professional (e.g., an importance rating), based on a timing of the request made by the telehealth professional (e.g., the oldest requests are the most important), based on a timing of when responses to the queries presented are needed by the telehealth professional, based on information determined by processor 20, based on the identity of the health professional, medical urgency (e.g. of following a certain treatment) according to state of the art in medicine and/or medical guidelines documents, based on the medical conditions of the patient, and based on patient abilities and preferences, and/or based on other information.

Electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., computing device 18, processor 20, etc.). In some embodiments, electronic storage 22 may be located in a server together with processor 20, in a server that is part of external resources 24, in computing device 18 associated with subject 12, and/or in other locations. Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information determined by processor 20, information received via computing device 18 and/or user interface 16 and/or other external computing systems, information received from external resources 24, information received from sensors 14, and/or other information that enables system 10 to function as described herein. By way of a non-limiting example, electronic storage 22 may store, responsive to levels of the one or more vital signs being outside the well-being ranges, information for a subject evaluation query interface withheld from the subject until the levels of the one or more vital signs are within the well-being ranges.

External resources 24 include sources of information (e.g., databases, websites, etc.), external entities participating with system 10 (e.g., a medical records system of a health care provider that stores psychological information and/or engagement information for subject 12), external home monitoring systems, one or more servers outside of system 10, a network (e.g., the internet), electronic storage, equipment related to Wi-Fi technology, equipment related to Bluetooth® technology, data entry devices, sensors, scanners, and/or other resources. In some implementations, some or all of the functionality attributed herein to external resources 24 may be provided by resources included in system 10. External resources 24 may be configured to communicate with processor 20, computing device 18, sensors 14, electronic storage 22, and/or other components of system 10 via wired and/or wireless connections, via a network (e.g., a local area network and/or the internet), via cellular technology, via Wi-Fi technology, and/or via other resources.

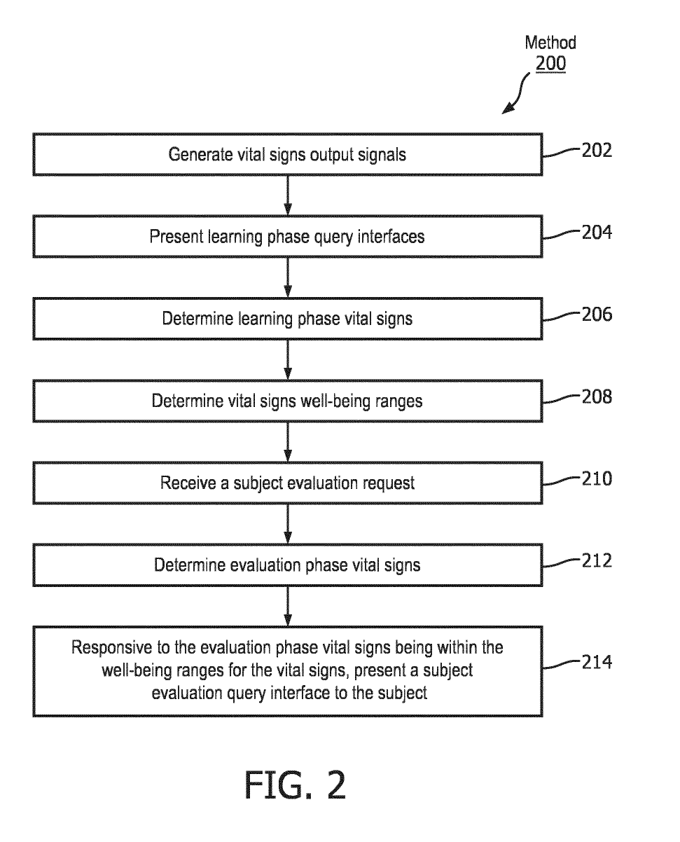

FIG. 2 illustrates a method 200 for facilitating interaction between a subject and a telemonitoring system. The system comprises one or more sensors, a user interface, one or more hardware processors, and/or other components. The one or more hardware processors are configured by machine readable instructions to execute computer program components. The computer program components include a learning phase component, a vital signs component, a range component, an evaluation phase component, and/or other components. The operations of method 200 presented below are intended to be illustrative. In some embodiments, method 200 may be accomplished with one or more additional operations not described, and/or without one or more of the operations discussed. Additionally, the order in which the operations of method 200 are illustrated in FIG. 2 and described below is not intended to be limiting.

In some embodiments, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, vital signs output signals are generated. In some embodiments, operation 202 is performed by sensors the same as or similar to sensors 14 (shown in FIG. 1 and described herein).

At an operation 204, learning phase query interfaces are presented to the subject. The learning phase query interfaces are presented via the user interface. The one or more learning phase query interfaces comprise queries related to a well-being of the subject and/or other queries. In some embodiments, operation 204 is performed by a processor component the same as or similar to learning phase component 32 (shown in FIG. 1 and described herein).

At an operation 206, learning phase vital signs are determined. In some embodiments, determining learning phase vital signs comprises determining, based on the output signals, first levels of the one or more vital signs for periods of time that correspond to a timing of responses to the queries of the one or more learning phase query interfaces by the subject. In some embodiments, operation 206 is performed by a processor component the same as or similar to vital signs component 34 (shown in FIG. 1 and described herein). At an operation 208, vital signs well-being ranges are determined. Well-being ranges may be determined for individual ones of the one or more vital signs of the subject. The well-being ranges for the one or more vital signs of the subject are determined based on the first levels of the one or more vital signs, information conveyed by the responses to the queries of the one or more learning phase query interfaces, and/or other information. In some embodiments, operation 208 is performed by a processor component the same as or similar to range component 36 (shown in FIG. 1 and described herein).

At an operation 210, a subject evaluation request is received. Receiving an evaluation request may include receiving a request from a telehealth professional for presentation of a subject evaluation query interface to the subject. The subject evaluation query interface comprises queries related to a medical condition experienced by the subject and/or other queries. In some embodiments, operation 210 is performed by a processor component the same as or similar to evaluation phase component 38 (shown in FIG. 1 and described herein).

At an operation 212, evaluation phase vital signs are determined. Determining evaluation phase vital signs includes determining, based on the output signals, second levels of the one or more vital signs of the subject. The second levels of the one or more vital signs of the subject may be current vital signs levels, for example. In some embodiments, operation 212 is performed by a processor component the same as or similar to vital signs component 34 (shown in FIG. 1 and described herein).

At an operation 214, responsive to the evaluation phase vital signs being within the well-being ranges for the vital signs, a subject evaluation query interface is presented to the subject. For example, responsive to the second levels of the one or more vital signs being within the well-being ranges, the subject evaluation query interface is presented to the subject via the user interface. In some embodiments, responsive to any of the second levels of the one or more vital signs being outside the well-being ranges, the subject evaluation query interface is withheld from the subject until the second levels of the one or more vital signs are within the well-being ranges. In some embodiments, operation 214 is performed by a processor component the same as or similar to evaluation phase component 38 (shown in FIG. 1 and described herein).

In some embodiments, method 200 includes obtaining psychological information associated with the subject (e.g., as part of operation 204), determining third levels of the one or more vital signs for periods of time that correspond to a timing of obtaining the psychological information (e.g., as part of operation 206), and determining the well-being ranges for the one or more vital signs of the subject based on the third levels of the one or more vital signs, and the obtained psychological information in addition to and/or instead of the information described above (e.g., as part of operation 208). In some embodiments, method 200 includes determining first engagement information indicative of a level of engagement of the subject with the telemonitoring system and second engagement information indicative of a level of engagement of the subject with a care team associated with the subject (e.g., as part of operation 204), determining fourth levels of the one or more vital signs for periods of time that correspond to a timing of determining the first and second engagement information (e.g., as part of operation 206), and determining the well-being ranges for the one or more vital signs of the subject based on the fourth levels of the one or more vital signs, and the first and second engagement information in addition to and/or instead of the information described above (e.g., as part of operation 208).

In some embodiments, method 200 further comprises re-determining, with the one or more hardware processors, the well-being ranges for the one or more vital signs (a) at predetermined daily, weekly, monthly, or yearly intervals; (b) responsive to a request by the telehealth professional; or (c) responsive to a predetermined change in the medical condition experienced by the subject.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A telemonitoring system configured to interact with a subject, the system comprising:
an input unit for receiving output signals, from one or more sensors, conveying information related to one or more vital signs of the subject;
a user interface configured to present the subject with one or more query interfaces and facilitate entry and/or selection of responses to queries presented by the query interfaces; and
one or more hardware processors configured by machine readable instructions to:
cause presentation of one or more learning phase query interfaces to the subject via the user interface, the one or more learning phase query interfaces comprising queries related to a well-being of the subject;
determine, based on the output signals, first levels of the one or more vital signs for periods of time that correspond to a timing of responses to the queries of the one or more learning phase query interfaces by the subject;
determine well-being ranges for the one or more vital signs of the subject based on the first levels of the one or more vital signs, and information conveyed by the responses to the queries of the one or more learning phase query interfaces;
receive a request for presentation of a subject evaluation query interface to the subject, the subject evaluation query interface comprising queries related to a medical condition experienced by the subject;
determine, based on the output signals, second levels of the one or more vital signs of the subject; and
responsive to the second levels of the one or more vital signs being within the well-being ranges, cause presentation of the subject evaluation query interface to the subject via the user interface.

2. The system of claim 1, wherein the one or more hardware processors are further configured to, responsive to any of the second levels of the one or more vital signs being outside the well-being ranges, withhold the subject evaluation query interface from the subject until the second levels of the one or more vital signs are within the well-being ranges.

3. The system of claim 1, wherein the one or more hardware processors are further configured to:
obtain psychological information associated with the subject;
determine, based on the output signals, third levels of the one or more vital signs for periods of time that correspond to a timing of obtaining the psychological information; and
determine the well-being ranges for the one or more vital signs of the subject based on the third levels of the one or more vital signs, and the obtained psychological information.

4. The system of claim 1, wherein the one or more hardware processors are further configured to:
determine first engagement information indicative of a level of engagement of the subject with the telemonitoring system and second engagement information indicative of a level of engagement of the subject with a care team associated with the subject;
determine, based on the output signals, fourth levels of the one or more vital signs for periods of time that correspond to a timing of determining the first and second engagement information; and
determine the well-being ranges for the one or more vital signs of the subject based on the fourth levels of the one or more vital signs, and the first and second engagement information.

5. The system of claim 1, wherein the one or more hardware processors are configured to re-determine the well-being ranges for the one or more vital signs (a) at predetermined daily, weekly, monthly, or yearly intervals; (b) responsive to a request by the telehealth professional; or (c) responsive to a predetermined change in the medical condition experienced by the subject.

6. The system of claim 1, comprising one or more sensors configured to generate the output signals conveying information related to the one or more vital signs of the subject.

7. A method for facilitating interaction between a subject and a telemonitoring system, an input unit and/or one or more sensors, a user interface and one or more hardware processors, the method comprising:
receiving output signals generated with the one or more sensors by conveying information related to one or more vital signs of the subject;
causing presentation, with the one or more hardware processors, of one or more learning phase query interfaces to the subject via the user interface, the one or more learning phase query interfaces comprising queries related to a well-being of the subject;
determining, with the one or more hardware processors based on the output signals, first levels of the one or more vital signs for periods of time that correspond to a timing of responses to the queries of the one or more learning phase query interfaces by the subject;
determining, with the one or more hardware processors, well-being ranges for the one or more vital signs of the subject based on the first levels of the one or more vital signs, and information conveyed by the responses to the queries of the one or more learning phase query interfaces;
receiving, with the one or more hardware processors, a request for presentation of a subject evaluation query interface to the subject, the subject evaluation query interface comprising queries related to a medical condition experienced by the subject;
determining, with the one or more hardware processors based on the output signals, second levels of the one or more vital signs of the subject; and
responsive to the second levels of the one or more vital signs being within the well-being ranges, causing, with the one or more hardware processors, presentation of the subject evaluation query interface to the subject via the user interface.

8. The method of claim 7, further comprising, responsive to any of the second levels of the one or more vital signs being outside the well-being ranges, withholding, with the one or more hardware processors, the subject evaluation query interface from the subject until the second levels of the one or more vital signs are within the well-being ranges.

9. The method of claim 7, further comprising:
obtaining, with the one or more hardware processors, psychological information associated with the subject;
determining, with the one or more hardware processors, based on the output signals, third levels of the one or more vital signs for periods of time that correspond to a timing of obtaining the psychological information; and
determining, with the one or more hardware processors, the well-being ranges for the one or more vital signs of the subject based on the third levels of the one or more vital signs, and the obtained psychological information.

10. The method of claim 7, further comprising:
determining, with the one or more hardware processors, first engagement information indicative of a level of engagement of the subject with the telemonitoring system and second engagement information indicative of a level of engagement of the subject with a care team associated with the subject;
determining, with the one or more hardware processors based on the output signals, fourth levels of the one or more vital signs for periods of time that correspond to a timing of determining the first and second engagement information; and
determining, with the one or more hardware processors, the well-being ranges for the one or more vital signs of the subject based on the fourth levels of the one or more vital signs, and the first and second engagement information.

11. The method of claim 7, further comprising:
re-determining, with the one or more hardware processors, the well-being ranges for the one or more vital signs (a) at predetermined daily, weekly, monthly, or yearly intervals; (b) responsive to a request by the telehealth professional; or (c) responsive to a predetermined change in the medical condition experienced by the subject.

12. The method of claim 7, further comprising:
generating, with the one or more sensors, the output signals conveying information related to one or more vital signs of the subject.

13. A telemonitoring system configured to interact with a subject, the system comprising:
means for receiving output signals conveying information related to one or more vital signs of the subject;
means for presenting the subject with one or more query interfaces and facilitating entry and/or selection of responses to queries presented by the query interfaces;
means for causing presentation of one or more learning phase query interfaces to the subject via the means for presenting, the one or more learning phase query interfaces comprising queries related to a well-being of the subject;
means for determining, based on the output signals, first levels of the one or more vital signs for periods of time that correspond to a timing of responses to the queries of the one or more learning phase query interfaces by the subject;
means for determining well-being ranges for the one or more vital signs of the subject based on the first levels of the one or more vital signs, and information conveyed by the responses to the queries of the one or more learning phase query interfaces;
means for receiving a request for presentation of a subject evaluation query interface to the subject, the subject evaluation query interface comprising queries related to a medical condition experienced by the subject;
means for determining, based on the output signals, second levels of the one or more vital signs of the subject; and
means for, responsive to the second levels of the one or more vital signs being within the well-being ranges, causing presentation of the subject evaluation query interface to the subject via the means for presenting.

14. The system of claim 13, further comprising means for, responsive to any of the second levels of the one or more vital signs being outside the well-being ranges, withholding the subject evaluation query interface from the subject until the second levels of the one or more vital signs are within the well-being ranges.

15. The system of claim 13, further comprising:
means for obtaining psychological information associated with the subject;
means for determining, based on the output signals, third levels of the one or more vital signs for periods of time that correspond to a timing of obtaining the psychological information; and
means for determining the well-being ranges for the one or more vital signs of the subject based on the third levels of the one or more vital signs, and the obtained psychological information.

16. The system of claim 13, further comprising:
means for determining first engagement information indicative of a level of engagement of the subject with the telemonitoring system and second engagement information indicative of a level of engagement of the subject with a care team associated with the subject;
means for determining, based on the output signals, fourth levels of the one or more vital signs for periods of time that correspond to a timing of determining the first and second engagement information; and
means for determining the well-being ranges for the one or more vital signs of the subject based on the fourth levels of the one or more vital signs, and the first and second engagement information.

17. The system of claim 13, further comprising means for re-determining the well-being ranges for the one or more vital signs (a) at predetermined daily, weekly, monthly, or yearly intervals; (b) responsive to a request by the telehealth professional; or (c) responsive to a predetermined change in the medical condition experienced by the subject.

18. The system of claim 13, further comprising means for generating the output signals conveying information related to one or more vital signs of the subject.

19. A telemonitoring system configured to interact with a subject, the system comprising one or more hardware processors configured by machine readable instructions to:
cause presentation of one or more learning phase query interfaces to the subject via a user interface, the one or more learning phase query interfaces comprising queries related to a well-being of the subject;
determine, based on output signals from one or more vital signs sensors, levels of one or more vital signs of the subject for periods of time that correspond to a timing of responses to the queries of the one or more learning phase query interfaces by the subject;
determine well-being ranges for the one or more vital signs of the subject based on the levels of the one or more vital signs determined for the periods of time that correspond to the timing of the responses to the queries of the one or more learning phase query interfaces, and information conveyed by the responses to the queries of the one or more learning phase query interfaces;
receive a request for presentation of a subject evaluation query interface to the subject, the subject evaluation query interface comprising queries related to a medical condition experienced by the subject;
determine, based on the output signals, current levels of the one or more vital signs of the subject; and
responsive to the current levels of the one or more vital signs being within the well-being ranges, cause presentation of the subject evaluation query interface to the subject via the user interface.
